(19) United States
(12) Patent Application Publication
(10) Pub. No.: US 2024/0383768 A1
(43) Pub. Date: Nov. 21, 2024

(54) MAGNETIC BEAD AND MAGNETIC BEAD REAGENT

(71) Applicant: SEIKO EPSON CORPORATION, TOKYO (JP)

(72) Inventor: Michio NAKAMORI, Matsumoto-shi (JP)

(21) Appl. No.: 18/665,867

(22) Filed: May 16, 2024

(30) Foreign Application Priority Data
May 16, 2023 (JP) 2023-080649

(51) Int. Cl. C01G 49/00; C01B 33/12

(52) U.S. Cl. CPC C01G 49/009; C01B 33/12; C01P 2004/61; C01P 2004/62; C01P 2006/12

(57) ABSTRACT

A magnetic bead includes: a magnetic metal particle; and a coating layer covering a surface of the magnetic metal particle and containing an inorganic oxide. A specific surface area A measured by a gas adsorption method is 0.3 m²/g or more and 10.0 m²/g or less, and a ratio A/B1 of the specific surface area A to a specific surface area B1 calculated based on a volume-based particle size distribution is 1.00 or more and 9.00 or less.

The present application is based on, and claims priority from JP Application Serial Number 2023-080649, filed May 16, 2023, the disclosure of which is hereby incorporated by reference herein in its entirety.

### BACKGROUND

### 1. Technical Field

The present disclosure relates to a magnetic bead and a magnetic bead reagent.

### 2. Related Art

In recent years, in the fields of diagnosis and biological science in the medical field, there is an increasing demand for examination of a biological substance. Among biological substance examination techniques, a polymerase chain reaction (PCR) method is a method for extracting a nucleic acid such as deoxyribonucleic acid (DNA) or ribonucleic acid (RNA), specifically amplifying and detecting the nucleic acid. In such a process of examining the biological substance, it is first necessary to extract a substance to be examined from a specimen. For the extraction of the biological substance, a magnetic separation method using magnetic beads is widely used. In the magnetic separation method, a biological substance is extracted by applying a magnetic field using magnetic beads having a function of carrying the biological substance to be extracted. Specifically, the magnetic beads having the function of carrying the substance to be examined on surfaces of the magnetic beads are dispersed in a dispersion medium, then the obtained dispersion liquid is attached to a magnetic field generation device such as a magnetic stand, and ON/OFF of magnetic field application is repeated a plurality of times. Accordingly, the substance to be examined is extracted. Since such a magnetic separation method is a method for separating and collecting the magnetic beads by a magnetic force, a rapid separation operation can be performed.

The same magnetic separation method is used not only in extraction by the PCR method but also in fields of protein purification, separation and extraction of exosomes and cells, and the like.

JP-A-9-19292 discloses a magnetic carrier for nucleic acid binding that is made of magnetic silica particles containing a superparamagnetic metal oxide and has a specific surface area of 100 m²/g to 800 m²/g. Since such a magnetic carrier for nucleic acid binding has a large specific surface area, a large amount of nucleic acids can be adsorbed non-specifically.

JP-A-9-19292 is an example of the related art.

The magnetic carrier for nucleic acid binding described in JP-A-9-19292 has a very large specific surface area and therefore strongly adsorbs the nucleic acids. Therefore, there is a concern that it takes time to elute the adsorbed nucleic acids.

When the specific surface area of the magnetic carrier for nucleic acid binding is large, a large amount of chemicals and impurities used for the nucleic acids before adsorption are incorporated. In this case, when the nucleic acids are eluted, the impurities are easily brought into an eluate, and there is a concern that purity of the extracted nucleic acids may decrease.

Therefore, it is a problem to obtain a magnetic bead that can efficiently extract a high-purity biological substance.

### SUMMARY

A magnetic bead according to an application example of the present disclosure includes:
a magnetic metal particle; and
a coating layer covering a surface of the magnetic metal particle and containing an inorganic oxide.

A specific surface area A measured by a gas adsorption method is 0.3 m²/g or more and 10.0 m²/g or less, and
a ratio A/B1 of the specific surface area A to a specific surface area B1 calculated based on a volume-based particle size distribution is 1.00 or more and 9.00 or less.

A magnetic bead reagent according to an application example of the present disclosure includes:
the magnetic bead according to the application example of the present disclosure; and
a dispersion medium in which the magnetic bead is dispersed.

### BRIEF DESCRIPTION OF THE DRAWINGS

FIG. 1 is a process flowchart showing an example of a biological substance extraction method.

FIG. 2 is a schematic diagram showing the biological substance extraction method shown in FIG. 1.

FIG. 3 is a schematic diagram showing the biological substance extraction method shown in FIG. 1.

FIG. 4 is a schematic diagram showing the biological substance extraction method shown in FIG. 1.

FIG. 5 is a schematic diagram showing the biological substance extraction method shown in FIG. 1.

FIG. 6 is a schematic diagram showing the biological substance extraction method shown in FIG. 1.

FIG. 7 is a cross-sectional view showing a magnetic bead according to an embodiment.

FIG. 8 is a cross-sectional view showing modification of the magnetic bead in FIG. 7.

### DESCRIPTION OF EMBODIMENTS

Hereinafter, a preferred embodiment of a magnetic bead and a magnetic bead reagent according to the present disclosure will be described in detail with reference to the accompanying drawings.

The magnetic bead according to the embodiment is a particle group which adsorbs a biological substance and which is used for magnetic separation. The magnetic separation is a technique of applying an external magnetic field to a container in which a solid phase containing magnetic beads and a liquid phase containing a dispersion medium are charged to magnetically attract the solid phase and thus separating the solid phase from the liquid phase.

The biological substance refers to substances such as nucleic acids such as DNA or RNA, proteins, saccharides, various cells such as cancer cells, peptides, bacteria, and viruses. The nucleic acid may be present in a state of being contained in, for example, a biological sample such as a cell or a biological tissue, a virus, or a bacterium. Such a biological substance is extracted through, for example, each step of dissolution, adsorption, washing, and elution, and is used for an examination or the like. By magnetically separating the magnetic bead where the biological substance is adsorbed, purification and extraction of the biological substance can be easily performed.

### 1. Biological Substance Extraction Method

Hereinafter, an example of a biological substance extraction method using magnetic separation will be described. In the following description, a case in which the biological substance is a nucleic acid will be described as an example.

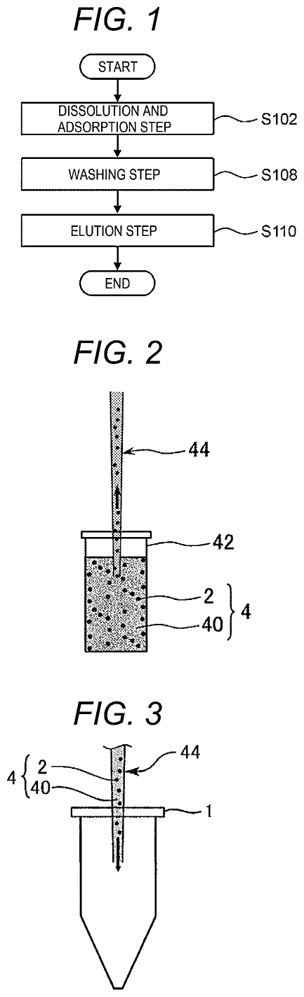

FIG. 1 is a process flowchart showing an example of the biological substance extraction method. FIGS. 2 to 6 are schematic diagrams showing the biological substance extraction method shown in FIG. 1.

The biological substance extraction method shown in FIG. 1 includes a dissolution and adsorption step S102, a washing step S108, and an elution step S110. Hereinafter, the steps will be sequentially described.

### 1.1. Dissolution and Adsorption Step

In the dissolution and adsorption step S102, first, as shown in FIG. 2, a magnetic bead dispersion liquid 4 (a magnetic bead reagent) containing magnetic beads 2 and a dispersion medium 40 is prepared in a container 42.

In the container 42, the magnetic bead dispersion liquid 4 is sufficiently stirred. Accordingly, the magnetic beads 2 are uniformly dispersed in the magnetic bead dispersion liquid 4. Thereafter, a predetermined amount of the magnetic bead dispersion liquid 4 is collected from the container 42 by a pipette 44 or the like and dispensed into a container 1 shown in FIG. 3. If a concentration distribution of the magnetic bead dispersion liquid 4 is uniform, a target amount of the magnetic beads 2 are added to the container 1 by the dispensing operation.

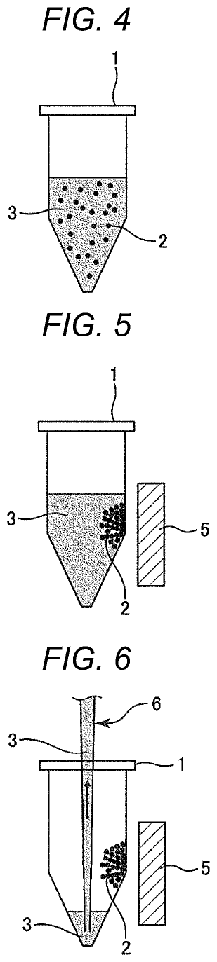

Next, a specimen sample, which contains a nucleic acid, and a dissolution and adsorption liquid are charged into the container 1 shown in FIG. 3. Then, contents in the container 1 are mixed. Accordingly, the magnetic beads 2 are dispersed in a liquid 3 in the container 1 as shown in FIG. 4. Since the nucleic acid is usually contained in a cell membrane and a nucleus, the nucleic acid is extracted by dissolving and removing the cell membrane and a so-called outer shell of the nucleus by a dissolution action of the dissolution and adsorption liquid. Thereafter, the nucleic acid is adsorbed to the magnetic beads 2 by an adsorption action of the dissolution and adsorption liquid.

As the dissolution and adsorption liquid, for example, a liquid containing a chaotropic substance is used. The chaotropic substance has a function of generating chaotropic ions in an aqueous solution, reducing an interaction between water molecules to destabilize a structure, and thus contributes to adsorption of the nucleic acid to the magnetic beads 2.

Particularly, when RNA among nucleic acids is extracted, it is preferable to acidify the liquid 3 in the container 1 by adding an acid or the like. An RNA monomer contains ribose and thus is more soluble in a polar solvent than DNA. When RNA and DNA are separated using such a difference, the liquid 3 may be acidified by, for example, adding an acid after the outer shell is dissolved and removed. Thereafter, a non-polar solvent such as phenol or chloroform is added to the liquid 3. Accordingly, DNA migrates to the non-polar solvent whereas RNA remains in the polar solvent. As a result, RNA and DNA can be separated, and RNA can be extracted.

When RNA is extracted, a pH of the liquid 3 in the container 1 is preferably 5.0 or less, more preferably 2.0 or more and 4.0 or less. Accordingly, an ionization equilibrium of a phosphate group contained in RNA is biased to a hydroxy group, and thus RNA is particularly easily dissolved in the polar solvent. Therefore, extraction efficiency of RNA can be further improved.

### 1.1.1. Magnetic Separation Operation

In the dissolution and adsorption step S102, an external magnetic field acts on the magnetic beads 2 to which the nucleic acid is adsorbed, and the magnetic beads 2 are magnetically attracted. Accordingly, the magnetic beads 2 are moved to an inner wall of the container 1 and fixed. As a result, as shown in FIG. 5, the magnetic beads 2 as the solid phase and the liquid 3 as the liquid phase can be separated. In the specification, an operation of fixing the magnetic beads 2 by applying such an external magnetic field is referred to as a “magnetic separation operation”.

Before the magnetic separation operation, the contents in the container 1 are stirred as necessary. Accordingly, a probability that the nucleic acid is adsorbed by the magnetic beads 2 increases. In the stirring, for example, a vortex mixer, hand shaking, or pipetting is used.

In the application of the external magnetic field, for example, a magnet 5 disposed beside the container is used. The magnet 5 may be an electromagnet or a permanent magnet. When the external magnetic field acts on the magnetic beads 2, the magnetic beads 2 move toward the magnet 5.

### 1.1.2. Liquid Discharge Operation

In the dissolution and adsorption step S102, in a state in which the magnetic beads 2 are fixed to the inner wall of the container 1, as shown in FIG. 6, the liquid 3 accumulated at a bottom of the container 1 is suctioned and discharged by, for example, a pipette 6. In the specification, such an operation of discharging the liquid 3 is referred to as a “liquid discharge operation”. By the liquid discharge operation, the magnetic beads 2 to which the nucleic acid is adsorbed remain in the container 1.

After the liquid discharge operation, an acceleration may be applied to the container 1 as necessary. Accordingly, the liquid 3 adhering to the magnetic beads 2 can be shaken off, and the unseparated liquid 3 can be reduced. The acceleration may be a centrifugal acceleration. A centrifugal separator may be used to apply the centrifugal acceleration.

### 1.2. Washing Step

In the washing step S108, the magnetic beads 2 to which the nucleic acid is adsorbed are washed. Washing refers to an operation of removing impurities by bringing the magnetic beads 2, to which the nucleic acid is adsorbed, into contact with a washing liquid and then separating the magnetic beads 2 from the washing liquid again in order to remove the impurities adsorbed on the magnetic beads 2.

Specifically, after the washing liquid is charged into the container 1 in which the magnetic beads 2, to which the nucleic acid is adsorbed, are charged, the magnetic separation operation and the liquid discharge operation are performed again.

Among these, in the magnetic separation operation, first, the washing liquid is supplied into the container 1 by a pipette or the like. Then, the magnetic beads 2 and the washing liquid are stirred. Accordingly, the washing liquid comes into contact with the magnetic beads 2, and the magnetic beads 2 to which the nucleic acid is adsorbed are washed. In the stirring, for example, a vortex mixer, hand shaking, or pipetting is used. At this time, the application of the external magnetic field may be temporarily turned off. Accordingly, since the magnetic beads 2 are redispersed in the washing liquid, washing efficiency can be further improved.

Next, as the liquid discharge operation, the washing liquid accumulated at the bottom of the container 1 is discharged in a state in which the magnetic beads 2 are fixed to the inner wall of the container 1. The magnetic beads 2 are washed by performing the above-described supply and discharge of the washing liquid at least once. Accordingly, the impurities can be removed with high accuracy.

The washing liquid is not particularly limited as long as the washing liquid is a liquid that does not promote elution of the nucleic acid and does not promote binding of the impurities to the magnetic beads 2. Examples of the washing liquid include organic solvents such as ethanol, isopropyl alcohol, and acetone, aqueous solutions of the organic solvents, and a low salt concentration aqueous solution.

The washing liquid may contain a surfactant such as Triton (registered trademark), Tween (registered trademark), or SDS. The washing liquid may contain a chaotropic substance such as guanidine hydrochloride.

The washing step S108 may be performed as necessary and may be omitted when washing is not necessary.

### 1.3. Elution Step

In the elution step S110, the nucleic acid adsorbed on the magnetic bead 2 is eluted into an elution liquid. The elution is an operation of shifting the nucleic acid to the elution liquid by bringing the magnetic bead 2, to which the nucleic acid is adsorbed, into contact with the elution liquid and then separating the magnetic bead 2 from the elution liquid again.

Specifically, after the elution liquid is charged into the container 1 in which the magnetic bead 2, to which the nucleic acid is adsorbed, is charged, the magnetic separation operation and the liquid discharge operation are performed again.

Among these, in the magnetic separation operation, first, the elution liquid is supplied into the container 1 by a pipette or the like. Then, the magnetic beads 2 and the elution liquid are stirred. Accordingly, the elution liquid comes into contact with the magnetic beads 2, and the nucleic acid is eluted into the elution liquid. In the stirring, for example, a vortex mixer, hand shaking, or pipetting is used. At this time, the application of the external magnetic field may be temporarily turned off. Accordingly, since the magnetic beads 2 are dispersed in the elution liquid, elution efficiency can be further improved.

Next, as the liquid discharge operation, the elution liquid accumulated at the bottom of the container 1 is discharged in a state in which the magnetic beads 2 are fixed to the inner wall of the container 1. Accordingly, the elution liquid containing the nucleic acid can be collected.

The elution liquid is not particularly limited as long as the elution liquid is a liquid that promotes the elution of the nucleic acid from the magnetic beads 2 to which the nucleic acid is adsorbed. For example, in addition to water such as sterilized water or pure water, a TE buffer solution, that is, an aqueous solution containing a 10 mM Tris-HCl buffer solution and 1 mM ethylenediaminetetraacetic acid (EDTA) and having a pH of about 8 is preferably used.

The elution liquid may contain a surfactant such as Triton (registered trademark), Tween (registered trademark), or SDS. In addition, the elution liquid may contain sodium azide as a preservative.

In the elution step S110, the elution liquid may be heated. Accordingly, the elution of the nucleic acid can be promoted. A heating temperature of the elution liquid is not particularly limited, and is preferably 70° C. or higher and 200° C. or lower, more preferably 80° C. or higher and 150° C. or lower, and still more preferably 95° C. or higher and 125° C. or lower.

### 2. Magnetic Bead

Next, the magnetic bead 2 will be described. The magnetic bead 2 is a particle that has magnetism and whose surface exhibits a binding affinity with a biological substance.

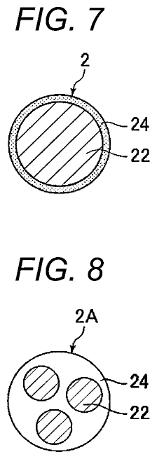

FIG. 7 is a cross-sectional view showing the magnetic bead 2 according to the embodiment. The magnetic bead 2 shown in FIG. 7 includes a magnetic metal particle 22 and a coating layer 24. A metal powder having magnetism is used as the magnetic metal particle 22. Therefore, the magnetic metal particle 22 has a high magnetization in a magnetic field and contributes to an improvement in a movement speed of the magnetic bead 2. Accordingly, a time required for the magnetic separation can be shortened.

Since the coating layer 24 contains an inorganic oxide, the coating layer 24 has a binding affinity with the biological substance. Since the coating layer 24 contains the inorganic oxide, the coating layer 24 has a high function of protecting the magnetic metal particle 22 from oxidation, corrosion, and the like. Therefore, elution of metal ions in the magnetic bead 2 can be prevented.

In the magnetic separation operation, a particle group that is an aggregate of such a magnetic bead 2 is used. In the specification, the magnetic bead 2 refers to the particle group or one particle constituting the particle group.

### 2.1. Characteristics of Magnetic Bead

### 2.1.1. Specific Surface Area

In the magnetic bead 2, when a specific surface area measured by a gas adsorption method is A and a specific surface area calculated based on a volume-based particle size distribution is B1, the specific surface area A and a specific surface area ratio A/B1 each satisfy a predetermined range to be described later.

Such a magnetic bead 2 can secure a sufficient amount of biological substance adsorbed per unit amount. Accordingly, the biological substance can be efficiently extracted. It is possible to prevent the adsorbed biological substance from being less likely to be eluted. Further, it is possible to prevent introduction of chemicals and impurities used in the respective steps described above, and it is possible to prevent a decrease in purity of the biological substance to be extracted. Therefore, according to such a magnetic bead 2, a high-purity biological substance can be efficiently extracted.

### 2.1.1.1. Specific Surface Area A Measured by Gas Adsorption Method

The specific surface area A of the magnetic beads 2 is measured, for example, according to a specific surface area measuring method for a powder (solid) by gas adsorption as defined in JIS Z 8830:2013. Examples of a device used for measuring the specific surface area include a specific surface area and pore size distribution measuring device, BELSORP-max-N—VP-CM, manufactured by MicrotracBEL. In addition, nitrogen or krypton may be used as a gas type used in the measurement.

The specific surface area A measured for the magnetic beads 2 is 0.3 m²/g or more and 10.0 m²/g or less. The specific surface area A is preferably 0.35 m²/g or more and 4.0 m²/g or less, more preferably 2.0 m²/g or less. When the specific surface area A is within the above range, a sufficient amount of biological substance adsorbed in a unit amount of the magnetic beads 2 can be secured.

When the specific surface area A is less than the lower limit value, the specific surface area A is insufficient, and thus the amount of biological substance adsorbed cannot be sufficiently secured. The magnetic beads 2 are likely to settle, and adsorption efficiency of the biological substance decreases. On the other hand, when the specific surface area A is more than the upper limit value, a large amount of chemicals and impurities other than the biological substance are adsorbed, leading to a decrease in purity of the biological substance to be finally extracted.

### 2.1.1.2. Specific Surface Area Ratio A/B1 Calculated Based on Volume-Based Particle Size Distribution

As described above, the specific surface area B1 of the magnetic beads 2 is a calculated value determined based on the volume-based particle size distribution of the magnetic beads 2. The calculated value is determined as follows.

First, for the magnetic beads 2, the volume-based particle size distribution is acquired, and an integrated distribution curve is determined based on the particle size distribution. In the obtained integrated distribution curve, a particle diameter at which a cumulative value a small diameter side is 10% is defined as a representative particle diameter D10 of the magnetic beads 2. Similarly, particle diameters at which cumulative values from the small diameter side are 20%, 30%, 40%, 50%, 60%, 70%, 80%, and 90% are referred to as representative particle diameters D20, D30, D40, D50, D60, D70, D80, and D90 of the magnetic beads 2. The volume-based particle size distribution is determined by a laser diffraction and dispersion method. Examples of a device for determining the particle size distribution by the laser diffraction and dispersion method include a particle size distribution measuring device MT3300 series manufactured by MicrotracBEL.

Next, a volume v and a surface area s are calculated for each of the particles with the representative particle diameters D10 to D90. At this time, the calculation is performed assuming that each particle is a sphere.

Subsequently, based on a density (a true specific gravity) of the magnetic beads 2, a volume V occupied by each of nine types of particle groups including particles with the representative particle diameters D10 to D90 constituting 1 g of the magnetic beads 2 is calculated. Here, it is assumed that the magnetic beads 2 are formed by mixing the nine types of particle groups with the representative particle diameters D10 to D90 in equal volumes. In this case, the nine types of particle groups are contained at the same volume ratio. The volume occupied by 1 g of the magnetic beads 2 is equally divided into the nine types of particle groups. For example, when the density of the magnetic beads 2 is 7 g/cm³, the occupied volume by each of the nine types of particle groups constituting 1 g of the magnetic beads 2 is 1/7 cm³/g. In this case, the volume V occupied by each of the nine types of particle groups in 1 g of the magnetic bead 2 is (1/7)/9 cm³/g.

Next, the volume V occupied by each of the nine types of particle groups constituting 1 g of the magnetic beads 2 is divided by the volume v of each of the nine types of particles calculated in advance. Accordingly, the number of particles n in each of the nine types of particle groups constituting 1 g of the magnetic beads 2 is determined.

Next, the surface area s of each of the nine types of particles calculated in advance is multiplied by the number of particles n in each of the nine types of particle groups constituting 1 g of the magnetic beads 2. Accordingly, a surface area S of each particle group constituting 1 g of the magnetic beads 2 is determined.

Next, an average value of the surface area S for each of the nine types of particle groups is calculated, and then the obtained average value is multiplied by nine. The calculation result is defined as the specific surface area B1. As described above, the specific surface area B1 of the magnetic bead 2 can be calculated.

In the magnetic bead 2 according to the embodiment, the ratio A/B1 of the specific surface area A to the specific surface area B1 calculated as described above is 1.00 or more and 9.00 or less. The ratio A/B1 is preferably 1.20 or more and 5.00 or less, and more preferably 1.50 or more and 3.00 or less. When the specific surface area ratio A/B1 is within the above range, the specific surface area A, which is a measured value, can be optimized with respect to the specific surface area B1 calculated based on the particle diameter of the magnetic bead 2. Accordingly, for example, a shape, such as a depth and narrowness, of pores of the coating layer 24 can be optimized. The shape of the pores depends on ease of elution of the adsorbed biological substance and ease of adsorption of chemicals and impurities other than the biological substance. Accordingly, when the specific surface area ratio A/B1 is within the above range, it is possible to simultaneously secure the ease of elution into the elution liquid while securing a sufficient amount of biological substance adsorbed to the magnetic bead 2. As a result, it is possible to obtain the magnetic bead 2 capable of extracting a sufficient amount of biological substance even when an elution time is relatively short. It is possible to prevent adsorption of chemicals or impurities other than the biological substance and to prevent a decrease in purity of the biological substance to be extracted.

When the specific surface area ratio A/B1 is less than the lower limit value, difficulty in producing the magnetic bead 2 increases. On the other hand, when the specific surface area ratio A/B1 is more than the upper limit value, the elution takes a long time, or when the elution time is short, a sufficient amount of biological substance cannot be eluted. In addition, chemicals and impurities other than the biological substance are also extracted, and the purity of the biological substance to be extracted decreases.

The specific surface area A can be adjusted according to a surface state of the magnetic metal particle 22, a production condition for the coating layer 24, and the like. For example, by employing a melting process, such as an annealing method, as a production method for the magnetic metal particle 22, surface smoothness can be improved, and the specific surface area A can be reduced without substantially changing the specific surface area B1. In this case, the ratio A/B1 can be reduced. Meanwhile, by increasing a film density of the coating layer 24, it is possible to reduce the specific surface area A without substantially changing the specific surface area B1. In this case, the ratio A/B1 can also be reduced.

In addition, the particle size distribution can be adjusted by classifying the produced metal powder. Accordingly, both the specific surface areas A and B1 can be adjusted.

### 2.1.1.3. Specific Surface Area Ratio A/B2 Calculated Based on Number-Based Particle Size Distribution

In the magnetic bead 2, when a specific surface area calculated based on a number-based particle size distribution is B2, a specific surface area ratio A/B2 preferably satisfies a predetermined range to be described later.

In such a magnetic bead 2, an effect of efficiently extracting a high-purity biological substance is more remarkable. The specific surface area B2 calculated based on the number-based particle size distribution is more influenced by fine particles than the specific surface area B1 described above. Therefore, even in a case of the particle size distribution in which a ratio of the fine particles is relatively large, extraction characteristics of the biological substance can be further optimized by evaluating the magnetic bead 2 using the specific surface area ratio A/B2.

The specific surface area B2 of the magnetic beads 2 is a calculated value determined based on the number-based particle size distribution of the magnetic beads 2. The calculated value is determined as follows.

First, for the magnetic beads 2, the number-based particle size distribution is acquired, and an integrated distribution curve is determined based on the particle size distribution. In the obtained integrated distribution curve, the representative particle diameters D10, D20, D30, D40, D50, D60, D70, D80, and D90 of the magnetic beads 2 are determined. The number-based particle size distribution is determined by a laser diffraction and dispersion method.

Next, in a manner same as the method for calculating the specific surface area ratio A/B1, the volume v and the surface area s are calculated for each of the particles with the representative particle diameters D10 to D90. At this time, the calculation is performed assuming that each particle is a sphere.

Next, the volume v of each particle is summed up to the representative particle diameters D10 to D90, and a total volume is determined. Further, a ratio r of the volume v of each particle to the total volume is calculated.

Subsequently, based on a density (a true specific gravity) of the magnetic beads 2, a volume V occupied by each of nine types of particle groups including particles with the representative particle diameters D10 to D90 constituting 1 g of the magnetic beads 2 is calculated. Here, it is assumed that the magnetic beads 2 are formed by mixing the nine types of particle groups with the representative particle diameters D10 to D90 at the above-described ratio r. The volume occupied by 1 g of the magnetic beads 2 is multiplied by the ratio r. For example, when the density of the magnetic beads 2 is 7 g/cm³, the occupied volume by each of the nine types of particle groups constituting 1 g of the magnetic beads 2 is 1/7 cm³/g. In this case, the volume V occupied by each of the nine types of particle groups in 1 g of the magnetic bead 2 is (1/7)×r cm³/g.

Next, the volume V occupied by each of the nine types of particle groups constituting 1 g of the magnetic beads 2 is divided by the volume v of each of the nine types of particles calculated in advance. Accordingly, the number of particles n in each of the nine types of particle groups constituting 1 g of the magnetic beads 2 is determined.

Next, the surface area s of each of the nine types of particles calculated in advance is multiplied by the number of particles n in each of the nine types of particle groups constituting 1 g of the magnetic beads 2. Accordingly, a surface area S of each particle group constituting 1 g of the magnetic beads 2 is determined.

Next, an average value of the surface area S for each of the nine types of particle groups is calculated, and then the obtained average value is multiplied by ten. The calculation result is defined as the specific surface area B2. As described above, the specific surface area B2 of the magnetic bead 2 can be calculated.

In the magnetic bead 2 according to the embodiment, the ratio A/B2 of the specific surface area A to the specific surface area B2 calculated as described above is 1.00 or more and 6.00 or less. The ratio A/B2 is preferably 1.10 or more and 3.00 or less, and more preferably 1.20 or more and 2.00 or less. When the specific surface area ratio A/B2 is within the above range, the specific surface area A, which is a measured value, can be optimized with respect to the specific surface area B2 calculated based on the particle diameter of the magnetic bead 2. Accordingly, for example, a shape, such as a depth and narrowness, of pores of the coating layer 24 can be more optimized in consideration of the presence of the fine particles. Therefore, when the specific surface area ratio A/B2 is within the above range, it is possible to implement the magnetic bead 2 capable of more efficiently extracting the biological substance with higher purity.

When the specific surface area ratio A/B2 is less than the lower limit value, the difficulty in producing the magnetic bead 2 may increase. On the other hand, when the specific surface area ratio A/B2 is more than the upper limit value, the elution takes a long time, or when the elution time is short, a sufficient amount of biological substance may not be eluted. In addition, chemicals and impurities other than the biological substance are also extracted, and the purity of the biological substance to be extracted may decrease.

### 2.1.2. Magnetic Characteristics

A saturation magnetization of the magnetic bead 2 is preferably 50 emu/g or more, more preferably 80 emu/g or more, and still more preferably 100 emu/g or more. The saturation magnetization is a magnetization value in a case in which a magnetization exhibited by a magnetic material when a sufficiently large magnetic field is externally applied is constant regardless of the magnetic field. When the saturation magnetization is within the above range, a function as a magnetic material can be sufficiently exhibited. Specifically, since a movement speed of the magnetic bead 2 in a magnetic field can be increased, a time required for magnetic separation can be shortened. The saturation magnetization of the magnetic bead 2 affects an adsorption force when the magnetic bead 2 is fixed by an external magnetic field. When the saturation magnetization is within the above range, a sufficiently high adsorption force can be obtained, and therefore, when the liquid 3 is discharged in a state in which the magnetic bead 2 is fixed, the magnetic bead 2 can be prevented from being discharged together with the liquid 3. Accordingly, it is possible to prevent a decrease in a biological substance yield due to a decrease in the number of the magnetic beads 2.

An upper limit value of the saturation magnetization of the magnetic bead 2 is not particularly limited, and is preferably 220 emu/g or less from the viewpoint of ease of selection of a material suitable for a balance between performance and cost.

The saturation magnetization of the magnetic bead 2 can be measured by a vibrating sample magnetometer (VSM) or the like. As the vibrating sample magnetometer, for example, TM-VSM1230-MHHL manufactured by Tamagawa Seisakusyo Co., Ltd. may be used. A maximum applied magnetic field when measuring the saturation magnetization is, for example, 0.5 T or more.

A coercive force Hc of the magnetic bead 2 is preferably 1500 A/m or less, more preferably 800 A/m or less, still more preferably 400 A/m or less, and particularly preferably 100 A/m or less. The coercive force Hc refers to a value of an external magnetic field in an opposite direction required to return a magnetized magnetic material to an unmagnetized state. That is, the coercive force Hc means a resistance force against an external magnetic field. As the coercive force Hc of the magnetic bead 2 decreases, the magnetic bead 2 is less likely to aggregate even when being switched from a state in which a magnetic field is applied to a state in which no magnetic field is applied, and the magnetic bead 2 can be uniformly dispersed in a dispersion liquid. Further, even when the switching of magnetic field application is repeated, since redispersibility of the magnetic bead 2 is improved as the coercive force Hc decreases, aggregation of the magnetic bead 2 can be further prevented. A lower limit value of the coercive force Hc of the magnetic bead 2 is not particularly limited, and is preferably 5 A/m or more from the viewpoint of ease of selection of a material suitable for a balance between performance and cost.

The coercive force Hc of the magnetic bead 2 can be measured by a vibrating sample magnetometer or the like in the same manner as the saturation magnetization described above. A maximum applied magnetic field when measuring the coercive force Hc is, for example, 15 kOe.

A relative permeability of the magnetic bead 2 is preferably 5 or more. When the relative permeability of the magnetic bead 2 is less than the lower limit value, responsiveness of the magnetic bead 2 decreases, and the time required for magnetic separation may be longer. An upper limit value of the relative permeability of the magnetic bead 2 is not particularly limited, and since the magnetic bead 2 is in a powder form, the relative permeability is usually substantially 100 or less due to an influence of a demagnetizing field.

### 2.1.3. Powder Characteristics

An average particle diameter D50 of the magnetic bead 2 is preferably 0.5 μm or more and 15 μm or less, more preferably 1.0 μm or more and 10 μm or less, still more preferably 1.5 μm or more and 8 μm or less, and particularly preferably 2 μm or more and 6 μm or less. When the average particle diameter D50 of the magnetic bead 2 is within the above range, the specific surface area of the magnetic bead 2 can be sufficiently large, and an attractive force and an adsorption force suitable for the magnetic separation can be generated in the magnetic bead 2. In addition, aggregation of the magnetic bead 2 can be prevented, and dispersibility can be improved. When the average particle diameter D50 of the magnetic bead 2 is less than the lower limit value, a magnetization value of the magnetic bead 2 is small, the magnetic bead 2 is likely to aggregate, and as a result, the biological substance extraction efficiency may decrease. In addition, the movement speed of the magnetic bead 2 may decrease, and the time required for the magnetic separation may increase. On the other hand, when the average particle diameter D50 of the magnetic bead 2 is more than the upper limit value, since the specific surface area of the magnetic bead 2 is small, a sufficient amount of the biological substance cannot be adsorbed, and an extraction amount of the biological substance may decrease. In addition, the magnetic beads 2 are likely to settle, the number of the magnetic beads 2 that can contribute to the extraction of the biological substance may decrease, and the biological substance extraction efficiency may decrease.

As for the average particle diameter D50 of the magnetic beads 2, a particle diameter (a median diameter) at which a cumulative value from a small diameter side is 50% in the integrated distribution curve obtained from the above volume-based particle size distribution is the average particle diameter D50 of the magnetic beads 2.

In the integrated distribution curve obtained from the volume-based particle size distribution, when a particle diameter at which the cumulative value from the small diameter side is 90% is defined as a 90% particle diameter D90, a ratio D90/D50 of the 90% particle diameter D90 to the average particle diameter D50 in the magnetic beads 2 is preferably 3.00 or less, more preferably 2.00 or less, and still more preferably 1.75 or less. Accordingly, since a content of coarse particles is low, it is possible to prevent the coarse particles from attracting relatively small surrounding particles and aggregating to form an aggregate. When an aggregate is formed, the aggregate settles due to own weight, which may cause a decrease in extraction efficiency and an increase in an examination time of the biological substance. Therefore, when the ratio D90/D50 is within the above range, occurrence of such a problem can be prevented. When the ratio D90/D50 is more than the upper limit value, the content of coarse particles is high, and therefore, even when the application of the external magnetic field is turned off, the dispersibility of the magnetic bead 2 decreases, and aggregation is likely to occur.

### 2.2. Configuration of Magnetic Bead

As described above, the magnetic bead 2 shown in FIG. 7 includes the magnetic metal particle 22 and the coating layer 24. Hereinafter, each part will be described.

### 2.2.1. Magnetic Metal Particle

The magnetic metal particle 22 is a metal particle having magnetism, and preferably contains at least one of Fe, Co, and Ni as an element. Accordingly, the magnetic bead 2 that has a high magnetization derived from the magnetic metal is obtained.

In particular, from the viewpoint of obtaining a high saturation magnetization, a composition of the magnetic metal particle 22 is preferably an alloy containing Fe as a main component (a Fe-based alloy). Specifically, an atomic ratio of Fe is more preferably 50% or more, and still more preferably 70% or more. Examples of the Fe-based alloy include a Fe—Co alloy, a Fe—Ni alloy, a Fe—Co—Ni alloy, and compounds containing Fe, Co, and Ni. From the viewpoint of obtaining a high magnetization, a carbonyl iron powder made of substantially 100 mass % of Fe may be used as the magnetic metal particle 22. According to such a Fe-based alloy, it is possible to obtain the magnetic metal particle 22 having a high saturation magnetization and a high permeability even when a particle diameter is small. Accordingly, it is possible to obtain the magnetic bead 2 whose movement speed due to the action of the external magnetic field is high and whose attractive force when captured by the external magnetic field is large. As a result, the time required for magnetic separation can be shortened, and the magnetic bead 2 can be prevented from being mixed into the elution liquid and becoming impurities.

The Fe-based alloy may contain one or two or more selected from the group consisting of Cr, Nb, Cu, Al, Mn, Mo, Si, Sn, B, C, P, Ti, and Zr according to target characteristics, in addition to an element exhibiting strong magnetism alone like Fe described above. Si is a main element in an alloy powder and is also an element that promotes amorphization.

The Fe-based alloy may contain impurities as long as an effect of the magnetic metal particle 22 is not impaired. The impurities in the embodiment are elements that are unintentionally mixed during production of a raw material of the magnetic metal particle 22 or the magnetic bead 2. The impurities are not particularly limited, and examples thereof include O, N, S, Na, Mg, and K.

An example of the Fe-based alloy is an alloy having a content of Si of preferably 1.0 atomic % or more and 30.0 atomic % or less, more preferably 1.5 atomic % or more and 14.0 atomic % or less, and still more preferably 2.0 atomic % or more and 7.0 atomic % or less. Since such an alloy has a high permeability, the saturation magnetization tends to be high.

The Fe-based alloy may contain at least one of B (boron) having a content of preferably 5.0 atomic % or more and 16.0 atomic % or less, more preferably 8.0 atomic % or more and 13.0 atomic % or less, and C (carbon) having a content of preferably 0.5 atomic % or more and 5.0 atomic % or less, more preferably 1.5 atomic % or more and 4.0 atomic % or less. These elements are elements that promote amorphization, and contribute to stably forming an amorphous structure or a nanocrystal structure in the magnetic metal particle 22. Accordingly, the magnetic metal particle 22 that has a low coercive force is obtained.

Further, the Fe-based alloy preferably contains chromium (Cr) having a content of 1.0 atomic % or more and 8.0 atomic % or less, more preferably 1.5 atomic % or more and 5.0 atomic % or less. Accordingly, corrosion resistance of the magnetic metal particle 22 can be improved. As a result, the magnetic metal particle 22 in which elution of metal ions is reduced is obtained.

A content of impurities is preferably 1.0 atomic % or less in total. At such a level, the effect of the magnetic metal particle 22 is not impaired even when impurities are contained.

The elements and the composition of the magnetic metal particle 22 can be identified by an ICP emission analysis method defined in JIS G 1258:2014, a spark emission analysis method defined in JIS G 1253:2002, or the like. When the magnetic metal particle 22 is covered with the coating layer 24, measurement can be performed by the above-described methods after the coating layer 24 is removed by a chemical or physical method. When it is difficult to remove the coating layer 24, for example, analysis can be performed, after cutting the magnetic bead 2, on a portion of the magnetic metal particle 22 that is a core with an analysis device such as an electron probe micro analyzer (EPMA) or energy dispersive X-ray spectroscopy (EDX).

A Vickers hardness of the magnetic metal particle 22 is preferably 100 or more, more preferably 300 or more, and still more preferably 800 or more. A method for measuring the hardness of the magnetic metal particle 22 is, for example, as follows. A plurality of particles of the magnetic metal particles 22 are taken out, embedded in a resin to prepare a resin-embedded sample, and then a cross section of each magnetic metal particle 22 is exposed at a surface of the resin-embedded sample through grinding and polishing. The cross section is subjected to indentation with a micro Vickers tester, a nanoindenter, or the like, and the hardness is measured based on a size of the indentation.

When the Vickers hardness of the magnetic metal particle 22 is less than the lower limit value, the magnetic metal particle 22 may be plastically deformed by an impact when the magnetic bead 2 undergoes collision. When plastic deformation occurs, the coating layer 24 may be peeled off or detached. An upper limit of the Vickers hardness is not particularly limited, and is preferably 3000 or less from the viewpoint of ease of selection of a material suitable for a balance of performance and cost.

A main metal structure constituting the magnetic metal particle 22 may take various forms such as a crystal structure, an amorphous structure, and a nanocrystal structure. An amorphous structure refers to a non-crystal structure in which no crystal is present, and a nanocrystal refers to a structure mainly formed of fine crystals having a crystal grain diameter of 100 nm or less. The amorphous structure and the nanocrystal structure impart a high hardness to the magnetic metal particle 22. When the structure is the amorphous structure or the nanocrystal structure, the coercive force Hc of the magnetic bead 2 has a particularly low value, which contributes to improvement in the redispersibility of the magnetic bead 2. A volume fraction of the magnetic metal particle 22 having an amorphous structure or a nanocrystal structure is preferably 40% or more, and more preferably 60% or more. The volume fraction is determined based on a result of crystal structure analysis by X-ray diffraction. Each of the crystal structure, the amorphous structure, and the nanocrystal structure may exist alone, or two or more thereof may co-exist.

The metal structure of the magnetic metal particle 22 can also be identified by crystal structure analysis by an X-ray diffraction method on the magnetic metal particle 22. Alternatively, the metal structure can be identified by analyzing a structure observation image or a diffraction pattern obtained with a transmission electron microscope (TEM) from a cut-out sample. For example, in the case of the amorphous structure, a diffraction peak derived from a metal crystal of an α-Fe phase or the like is not observed in peak analysis of the X-ray diffraction method. In the case of the amorphous structure, a so-called halo pattern is formed in an electron diffraction pattern by TEM, and formation of a spot due to a crystal is not observed. The nanocrystal structure is formed of a crystal structure having a grain size of, for example, 100 nm or less, and can be checked from a TEM observation image. More accurately, an average grain diameter can be calculated by image processing or the like from a plurality of TEM structure observation images in which a plurality of crystals are present. In addition, the crystal grain diameter can be estimated by a Scherrer method based on a diffraction peak of a target crystal phase obtained by an X-ray diffraction method. Further, for a crystal structure having a large grain diameter, the crystal grain diameter can be measured by a method such as observing a cross section with an optical microscope or a scanning electron microscope (SEM).

In order to obtain the amorphous structure and the nanocrystal structure, it is effective to increase a cooling rate at the time of cooling after a molten raw material is pulverized when producing the magnetic metal particle 22. Ease of forming the amorphous structure and the nanocrystal structure also depends on an alloy composition. As a specific alloy system suitable for forming the amorphous structure and the nanocrystal structure, a composition in which one or two or more selected from the group consisting of Cr, Si, B, C, P, Nb, and Cu are added to Fe is preferable.

The magnetic metal particle 22 is produced by a method according to a general metal powder production method. Examples of the production method include a melting process in which a metal is melted, solidified, and powdered, a chemical process in which a powder is produced by a reduction method or a carbonyl method, and a mechanical process in which a metal having a larger shape such as an ingot is mechanically pulverized to obtain a powder. Among these, the melting process is suitable for producing the magnetic metal particle 22.

Among production methods based on the melting process, a representative production method is an atomization method. The atomization method causes a molten metal to collide with a fluid (liquid or gas) injected at a high speed, be rapidly solidified to form a powder, and is classified into a water atomization method, a high-pressure water atomization method, a rotary water jet atomization method, a gas atomization method, and the like depending on a difference in a type of a cooling medium or a device configuration. According to the atomization method, the magnetic metal particle 22 can be efficiently produced. Further, in the high-pressure water atomization method, the rotary water jet atomization method, and the gas atomization method, a particle shape of the metal powder is closer to a spherical shape due to an action of surface tension.

In particular, spheroidization during rapid solidification can be promoted by increasing a heating temperature of the molten metal to a temperature higher than a melting point of the raw material, preferably 100° C. or higher, and more preferably 150° C. or higher. The specific surface area of the magnetic metal particles 22 can be reduced by the spheroidization. As a result, the specific surface area A of the magnetic bead 2 can be reduced.

### 2.2.2. Coating Layer

As shown in FIG. 7, the coating layer 24 covers a particle surface of the magnetic metal particle 22. The coating layer 24 may be formed at at least a part of the particle surface of the magnetic metal particle 22, and preferably covers the entire particle surface.

The coating layer 24 covers the particle surface of the magnetic metal particle 22 and contains an inorganic oxide. Examples of the inorganic oxide include a silicon oxide, a magnesium oxide, a calcium oxide, an aluminum oxide, a titanium oxide, a zirconium oxide, a boron oxide, and an yttrium oxide, and a mixture of one or two or more thereof may be used. Since the coating layer 24 containing such an inorganic oxide is porous, a specific surface area is increased. Accordingly, an amount of biological substance adsorbed to the coating layer 24 can be increased.

The inorganic oxide is particularly preferably a silicon oxide. The silicon oxide specifically adsorbs the biological substance such as a nucleic acid in the dissolution and adsorption liquid, thereby enabling efficient extraction and collection of the biological substance. Further, since the silicon oxide is chemically stable, oxidation and corrosion of the magnetic metal particle 22 can be particularly prevented, and the corrosion resistance of the magnetic bead 2 can be particularly improved.

The silicon oxide is represented by a composition formula of SiOₓ (0<x≤2), and is preferably SiO₂. The silicon oxide may form a composite oxide or a composite with one or two or more selected from the group consisting of Al, Ti, V, Nb, Cr, Mn, Sn, and Zr.

The inorganic oxide may contain a substance (impurities) other than the inorganic oxide within a range in which the above-described effects are not impaired, for example, at a ratio of preferably 50 mass % or less, more preferably 10 mass % or less of the above-described inorganic oxide. When the silicon oxide is used as the inorganic oxide, examples of the impurities include C, N, and P.

A composition of the inorganic oxide can be checked by, for example, EDX analysis or Auger electron spectroscopy.

An average thickness of the coating layer 24 is preferably 10 nm or more, more preferably 20 nm or more, and still more preferably 30 nm or more. Accordingly, even when the magnetic bead 2 collides with each other or collides with an inner wall of the container or the like, the coating layer 24 can be prevented from being broken or peeled off. As a result, elution of metal ions and the like due to exposure of the magnetic metal particle 22 can be prevented. Meanwhile, the average thickness of the coating layer 24 is preferably 500 nm or less, more preferably 200 nm or less, and still more preferably 150 nm or less. Accordingly, a decrease in a magnetization per volume of the magnetic bead 2 can be prevented, and a decrease in the movement speed of the magnetic bead 2 can be prevented.

When the average thickness of the coating layer 24 is t and the average particle diameter of the magnetic beads 2 is D50, a ratio t/D50 of t to D50 is preferably 0.0005 or more and 0.2 or less, more preferably 0.001 or more and 0.05 or less, and still more preferably 0.005 or more and 0.03 or less. When t/D50 is less than the lower limit value, a ratio of the thickness of the coating layer 24 to the size of the magnetic metal particle 22 is excessively small, and thus the coating layer 24 may be broken or peeled off when the magnetic bead 2 collides with each other or the magnetic bead 2 collides with the inner wall of the container 1 or the like. Therefore, an amount of the biological substance adsorbed and extracted by the surface of the coating layer 24 decreases, and the extraction efficiency may decrease. When there are fragments of the peeled coating layer 24 or the magnetic metal particle 22 in an extracted liquid, the fragments may be mixed as impurities (contamination) at the same time when the biological substance is extracted. Further, the magnetic metal particle 22 may be exposed due to breaking and peeling of the coating layer 24, and elution of metal ions or the like may occur when the magnetic metal particle 22 comes into contact with an acidic solution or the like, resulting in a decrease in the biological substance extraction efficiency. On the other hand, when t/D50 is more than the upper limit value, a volume ratio of the coating layer 24 to an entire volume of the magnetic bead 2 is large, and a magnetization per volume of the magnetic bead 2 may decrease. Accordingly, the movement speed when the external magnetic field acts on the magnetic bead 2 decreases, and the time required for magnetic separation may increase.

The thickness of the coating layer 24 can be measured from, for example, a cross-sectional observation image of the magnetic bead 2 observed by a transmission electron microscope or a scanning electron microscope. The average thickness t of the coating layer 24 can be calculated by acquiring a plurality of observation images and averaging measured values: from image processing or the like. For example, the average thickness t is a value obtained by measuring the thickness of the coating layer 24 at five or more positions for one magnetic bead 2, determining an average value thereof, and then averaging the average value for ten or more magnetic beads 2. For example, intensities of a Si—K characteristic X-ray and a Fe-L characteristic X-ray may be compared using an analysis device such as energy dispersive X-ray spectroscopy (EDX), and the thickness of the coating layer 24 may be calculated based on a comparison result. That is, as will be described later, when the coating layer 24 contains silicon and the magnetic metal particle 22 includes a Fe-based alloy, an intensity ratio of a Si—K characteristic X-ray derived from the coating layer 24 to a sum of a Fe-L characteristic X-ray derived from the magnetic metal particle 22 and a Si—K characteristic X-ray derived from the coating layer 24 can be converted into the thickness of the coating layer 24.

Examples of a method for forming the coating layer 24 include a wet formation method such as a sol-gel method and a dry formation method such as a vapor-phase deposition method. Among these, a Stober method, which is a type of the sol-gel method, or an atomic layer deposition (ALD) method can be preferably used. The Stober method is a method for forming monodisperse particles by hydrolysis of a metal alkoxide. For example, when the coating layer 24 is formed of the silicon oxide, the silicon oxide can be produced by a hydrolysis reaction of a silicon alkoxide. Before the coating layer 24 is formed, a base thereof, for example, a surface of a particle of the magnetic metal particle 22 may be subjected to a washing treatment using water or an organic solvent.

Hereinafter, an example of a film formation condition for the coating layer 24 by the Stober method using a silicon alkoxide will be described.

An addition rate of the silicon alkoxide is, for example, preferably a rate of 0.00010 mol/h or more and 0.00100 mol/h or less, and more preferably a rate of 0.00016 mol/h or more and 0.00064 mol/h or less, in terms of per 1 m² of a surface area of the magnetic metal particle 22. With such an addition rate, it is possible to form a silicon oxide coating having a higher density and a smaller specific surface area A.

A deposition rate of the silicon oxide coating is preferably 1 nm/h or more and 50 nm/h or less, and more preferably 5 nm/h or more and 20 nm/h or less. With such a deposition rate, it is possible to more efficiently form a silicon oxide coating having a higher density and a smaller specific surface area A.

A density of the silicon oxide coating is not particularly limited, and is preferably 1.7 g/cm3 or more and 2.2 g/cm3 or less, and more preferably 1.8 g/cm3 or more and 2.1 g/cm3 or less. By setting the density of the silicon oxide coating within the above range, the specific surface area ratios A/B1 and A/B2 can be easily kept within the above range. That is, it is possible to form the coating layer 24 in which the shape and the density of the pores are optimized.

The coating layer 24 may cover a surface of one magnetic metal particle 22, or may cover a plurality of magnetic metal particles 22 together.

FIG. 8 is a cross-sectional view showing a modification of the magnetic bead 2 in FIG. 7.

A magnetic bead 2A shown in FIG. 8 contains a plurality of magnetic metal particles 22. The coating layer 24 covers the plurality of magnetic metal particles 22. With such a magnetic bead 2A, the effects same as those of the magnetic bead 2 shown in FIG. 7 can still be obtained.

The number of the magnetic metal particles 22 of the magnetic bead 2A is not particularly limited, and is 2 or more and 100 or less.

### 3. Magnetic Bead Dispersion Liquid

Next, the magnetic bead dispersion liquid 4 according to the embodiment will be described. As described above, the magnetic bead dispersion liquid 4 shown in FIG. 2 contains the magnetic bead 2 and the dispersion medium 40.

Examples of the dispersion medium 40 include polar organic solvents such as water, saltwater, and alcohols, and aqueous solutions thereof. Examples of water include sterilized water and pure water. Examples of alcohols include ethanol and isopropyl alcohol.

A concentration of the magnetic bead 2 in the magnetic bead dispersion liquid 4 is not particularly limited as long as sufficient uniformity can be secured by stirring the magnetic bead dispersion liquid 4.

A surfactant may be added for the purpose of improving the dispersibility of the magnetic bead 2 in the magnetic bead dispersion liquid 4. Examples of the surfactant include a non-ionic surfactant, a cationic surfactant, an anionic surfactant, and an amphoteric surfactant.

Examples of the non-ionic surfactant include Triton (registered trademark) series surfactants such as Triton-X, Tween (registered trademark) series surfactants such as Tween 20, and acyl sorbitan. Examples of the cationic surfactant include dodecyltrimethylammonium bromide, dodecyltrimethylammonium chloride, and cetyltrimethylammonium bromide. Examples of the anionic surfactant include sodium dodecyl sulfate (SDS), sodium N-lauroylsarcosinate, sodium cholate, sodium lauryl sulfate, and sarcosine. An example of the amphoteric surfactant is phosphatidylethanolamine. These surfactants are each used alone or two or more thereof are used in combination.

A content of the surfactant in the magnetic bead dispersion liquid 4 is preferably equal to or higher than a critical micelle concentration of the surfactant. The critical micelle concentration is also referred to as cmc and refers to a concentration where molecules of a surfactant dispersed in a liquid aggregate to form a micelle. When the content of the surfactant is equal to or higher than the critical micelle concentration, the surfactant is likely to form a layer around the magnetic bead 2. Accordingly, an effect of preventing aggregation of the magnetic bead 2 can be further improved.

The content of the surfactant is not limited to be equal to or higher than the critical micelle concentration, and may be less than the critical micelle concentration. For example, the content of the surfactant in the magnetic bead dispersion liquid 4 is preferably 0.05 mass % or more and 3.0 mass % or less regardless of the critical micelle concentration.

Further, it is preferable to add a preservative in order to impart long-term storage stability and a preservation effect to the magnetic bead dispersion liquid 4. An example of the preservative is sodium azide. A concentration of the preservative added is preferably 0.02 mass % or more and less than 0.1 mass % of the magnetic bead dispersion liquid 4. When the content is less than 0.02 mass %, a sufficient effect for long-term storage stability and preservation may not be obtained, and when the content is 0.1 mass % or more, problems such as lowering the biological substance extraction efficiency may occur.

A buffer may be added for the purpose of pH adjustment. An example of the buffer is Tris buffer.

### 4. Advantages Exerted by Embodiment

As described above, the magnetic bead 2 according to the embodiment includes the magnetic metal particle 22 and the coating layer 24. The coating layer 24 covers the surface of the magnetic metal particle 22 and contains an inorganic oxide. Further, when the specific surface area of the magnetic bead 2 measured by the gas adsorption method is A, and the specific surface area calculated based on the volume-based particle size distribution is B1, the magnetic bead 2 has the specific surface area A is 0.3 m²/g or more and 10.0 m²/g or less, and the ratio A/B1 of the specific surface area A to the specific surface area B1 is 1.00 or more and 9.00 or less.

According to such a configuration, since the specific surface area A is optimized, a sufficient amount of biological substance adsorbed in a unit amount of the magnetic bead 2 can be secured. Since the ratio A/B1 is also optimized, it is possible to simultaneously secure the ease of elution into the elution liquid while securing a sufficient amount of biological substance adsorbed to the magnetic bead 2. As a result, it is possible to obtain the magnetic bead 2 capable of extracting a sufficient amount of biological substance even when an elution time is relatively short. It is possible to prevent adsorption of chemicals or impurities other than the biological substance and to prevent a decrease in purity of the biological substance to be extracted. Therefore, according to the above configuration, the magnetic bead 2 capable of efficiently extracting a high-purity biological substance can be obtained.

The inorganic oxide is preferably a silicon oxide.

Since the silicon oxide is chemically stable, oxidation and corrosion of the magnetic metal particle 22 can be particularly prevented, and the corrosion resistance of the magnetic bead 2 can be particularly improved. The silicon oxide specifically adsorbs the biological substance such as a nucleic acid in the dissolution and adsorption liquid, thereby enabling efficient extraction and collection of the biological substance.

The ratio A/B2 of the specific surface area A to the specific surface area B2 calculated based on the number-based particle size distribution is preferably 1.00 or more and 6.00 or less.

According to such a configuration, the magnetic bead 2 capable of more efficiently extracting a biological substance with higher purity can be obtained.

The magnetic metal particle 22 is preferably made of a Fe-based alloy.

According to such a configuration, it is possible to obtain the magnetic metal particle 22 having a high saturation magnetization and a high permeability even when the particle diameter is small. Accordingly, it is possible to obtain the magnetic bead 2 whose movement speed due to the action of the external magnetic field is high and whose attractive force when captured by the external magnetic field is large. As a result, the time required for magnetic separation can be shortened, and the magnetic bead 2 can be prevented from being mixed into the elution liquid and becoming impurities.

The Fe-based alloy preferably includes an amorphous structure.

According to such a configuration, the magnetic bead 2 that has a low coercive force and excellent redispersibility can be obtained.

The particle diameter D50 at which a cumulative value from a small diameter side in an integrated distribution curve obtained from a volume-based particle size distribution is 50% is preferably 0.5 μm or more and 15 μm or less.

According to such a configuration, the specific surface area of the magnetic bead 2 can be sufficiently large, and an attractive force and an adsorption force suitable for the magnetic separation can be generated in the magnetic bead 2. In addition, aggregation of the magnetic bead 2 can be prevented, and dispersibility can be improved.

The ratio t/D50 of the average thickness t of the coating layer 24 to the particle diameter D50 is preferably 0.0005 or more and 0.2 or less.

According to such a configuration, peeling or the like of the coating layer 24 due to collision of the magnetic bead 2 can be prevented. Elution of metal ions and the like can be prevented. Further, a decrease in the magnetization per volume of the magnetic bead 2 can be prevented. As a result, the magnetic separation can be performed more rapidly, and the magnetic bead 2 having higher biological substance extraction efficiency is obtained.

The magnetic bead dispersion liquid 4 as the magnetic bead reagent according to the embodiment contains the magnetic bead 2 and the dispersion medium 40 in which the magnetic bead 2 is dispersed.

According to such a configuration, since the magnetic metal particle 22 has a high magnetization derived from the magnetic metal, it is possible to obtain the magnetic bead dispersion liquid 4 that has a high movement speed under the action of the external magnetic field and can rapidly perform the magnetic separation. Since the metal ion concentration of the supernatant obtained by the above operations is optimized, the magnetic bead dispersion liquid 4 that has high biological substance extraction efficiency is obtained.

The magnetic bead and the magnetic bead reagent according to the present disclosure are described above based on the shown embodiment, but the present disclosure is not limited thereto. For example, the magnetic bead and the magnetic bead reagent according to the present disclosure may be obtained by adding any component to the embodiment.

### EXAMPLES

Next, specific examples according to the present disclosure will be described.

### 5. Preparation of Magnetic Bead

First, magnetic metal particles made of soft magnetic materials A to D having compositions shown in Table 1 were prepared by a high-pressure water atomization method. A metal structure of the obtained magnetic metal particles was analyzed by an X-ray diffraction method. The metal structure identified by the analysis is shown in Table 1. Soft magnetic materials E and F are ferrite (non-metal soft magnetic material).

TABLE 1

| Soft | | | |
|---|---|---|---|
| magnetic | | Composition formula | Density |
| material | Metal structure | (atomic ratio) | (g/cm³) |
| A | Non-crystalline | Fe₈₁Si₅B₁₂C₂ | 6.82 |
| B | Non-crystalline | Fe₇₃Si₁₁Cr₂B₁₁C₃ | 6.52 |
| C | Non-crystalline | Fe_{73.5}Si_{13.5}CU₁B₉Nb₃ | 6.66 |
| D | Crystalline | Fe_{88.6}Si_{6.7}Cr_{4.7} | 7.47 |
| E | Non-metal | Ferrite | 3.54 |
| F | Non-metal | Ferrite | 1.50 |

Next, a silicon oxide (SiO₂) was formed into a film at a surface of the magnetic metal particle by a Stober method to obtain a coating layer. In the Stober method, first, 100 g of a magnetic metal powder was dispersed in 950 mL of ethanol and mixed, and the mixed liquid was stirred for 20 minutes by an ultrasonic application device. After the stirring, a mixed solution of 30 mL of pure water and 180 mL of ammonia water was added, followed by stirring for 10 minutes. Thereafter, a mixed liquid of tetraethoxysilane (TEOS) and 100 mL of ethanol was further added and stirred to form the coating layer at the surface of the magnetic metal particle. A formation time in this case was 30 minutes, and the formation time was changed according to a thickness to be formed. An addition rate of TEOS in this case was adjusted as appropriate within a range of 0.00016 mol/h or more and 0.00064 mol/h or less. Further, a deposition rate of the coating layer was adjusted as appropriate within a range of 5 nm/h or more and 20 nm/h or less. Thereafter, the obtained coating layer was washed with ethanol and acetone, respectively. After the washing, the resultant was dried at 65° C. for 30 minutes and further heated at 200° C. for 90 minutes. Accordingly, magnetic beads in samples Nos. 1 to 18 shown in Tables 2 and 3 were obtained.

A saturation magnetization and a coercive force of the magnetic bead are shown in Tables 2 and 3.

### 6. Measurement of Specific Surface Area A

For the magnetic beads of samples Nos. 1 to 18, the specific surface area A was measured by the above-described method. The measurement results are shown in Tables 2 and 3.

In Tables 2 and 3, magnetic beads corresponding to the present disclosure are each indicated by “Example”, and magnetic beads not corresponding to the present disclosure are each indicated by “Comparative Example”.

### 7. Calculation of Specific Surface Areas B1 and B2

For the magnetic beads of Sample Nos. 1 to 18, the volume-based particle size distribution and the number-based particle size distribution were measured. For the measurement, a particle size distribution measuring device MT3300 series manufactured by MicrotracBEL was used. Subsequently, based on the obtained particle size distribution and the density shown in Table 1, the specific surface areas B1 and B2 were calculated by the above-described calculation method. The ratio A/B1 of the specific surface area A to the specific surface area B1 and the ratio A/B2 of the specific surface area A to the specific surface area B2 were calculated. The calculation results are shown in Tables 2 and 3.

TABLE 2

| | | Configuration of magnetic bead | | | | | | |
|---|---|---|---|---|---|---|---|---|
| | | Magnetic | | | | | | |
| | | particle | Average | Coating film | | | Magnetic | |
| | | Soft | particle | | Average | | characteristics | |
| | | magnetic | diameter | Constituent | thickness | | Saturation | Coercive |
| Sample | Classifi- | material | D50 | material | t | t/D50 | magnetization | force |

| No. | cation | — | μm | — | nm | — | emu/g | A/m | Oe |
|---|---|---|---|---|---|---|---|---|---|
| 1 | Examples | A | 3.6 | Silicon | 30 | 0.0083 | 151 | 24 | 0.3 |
| | | | | oxide | | | | | |
| 2 | Examples | A | 2.5 | Silicon | 30 | 0.0120 | 151 | 24 | 0.3 |
| | | | | oxide | | | | | |
| 3 | Examples | A | 1.5 | Silicon | 30 | 0.0200 | 151 | 24 | 0.3 |
| | | | | oxide | | | | | |
| 4 | Examples | A | 0.8 | Silicon | 30 | 0.0375 | 151 | 24 | 0.3 |
| | | | | oxide | | | | | |
| 5 | Comparative | F | 4.6 | Silicon | — | — | 22 | 957 | 12.0 |
| | Example | | | oxide | | | | | |
| 6 | Comparative | F | 1.1 | Silicon | — | — | 35 | 718 | 9.0 |
| | Example | | | oxide | | | | | |
| 7 | Comparative | A | 6.0 | Silicon | 2 | 0.0003 | 25 | 1196 | 15.0 |
| | Example | | | oxide | | | | | |
| 8 | Comparative | A | 6.0 | Silicon | 250 | 0.0417 | 25 | 1196 | 15.0 |
| | Example | | | oxide | | | | | |

| | Configuration of magnetic bead | | | | |
|---|---|---|---|---|---|
| | Specific surface area | | | | |
| | | Specific | | Specific | |
| | Specific | surface area | | surface area | |
| | surface | B1 | | B2 | |
| | area A | calculated | | calculated | |
| | measured | based on | | based on | |
| | by gas | volume-based | | number-based | |
| | adsorption | particle size | Ratio | particle size | |
| Sample | method | distribution | A/B1 | distribution | Ratio |
| No. | m²/g | m²/g | — | m²/g | A/B2 |
| 1 | 0.40 | 0.24 | 1.67 | 0.33 | 1.21 |
| 2 | 0.57 | 0.34 | 1.68 | 0.48 | 1.19 |
| 3 | 0.95 | 0.56 | 1.70 | 0.84 | 1.13 |
| 4 | 1.77 | 0.85 | 2.08 | 1.50 | 1.18 |
| 5 | 18.34 | 0.36 | 50.94 | 1.28 | 14.33 |
| 6 | 20.05 | 4.67 | 4.29 | 3.38 | 5.93 |
| 7 | 0.29 | 0.20 | 1.45 | 0.27 | 1.07 |
| 8 | 25.00 | 0.20 | 125.00 | 0.27 | 92.59 |

TABLE 3

| | | Configuration of magnetic bead | | | | | | | |
|---|---|---|---|---|---|---|---|---|---|
| | | Magnetic | | | | | | | |
| | | particle | Average | Coating film | | | Magnetic | | |
| | | Soft | particle | | Average | | characteristics | | |
| | | magnetic | diameter | Constituent | thickness | | Saturation | Coercive | |
| Sample | Classifi- | material | D50 | material | t | t/D50 | magnetization | force | |
| No. | cation | — | μm | — | nm | — | emu/g | A/m | Oe |
| 9 | Examples | B | 4.8 | Silicon | 150 | 0.00313 | 151 | 24 | 0.3 |
| | | | | oxide | | | | | |
| 10 | Examples | B | 1.8 | Silicon | 10 | 0.0056 | 151 | 24 | 0.3 |
| | | | | oxide | | | | | |
| 11 | Examples | C | 5.6 | Silicon | 70 | 0.0125 | 119 | 80 | 1.0 |
| | | | | oxide | | | | | |
| 12 | Examples | C | 1.3 | Silicon | 150 | 0.1154 | 119 | 80 | 1.0 |
| | | | | oxide | | | | | |
| 13 | Examples | D | 7.8 | Silicon | 20 | 0.0026 | 185 | 797 | 10.0 |
| | | | | oxide | | | | | |
| 14 | Examples | D | 3.2 | Silicon | 30 | 0.0094 | 185 | 797 | 10.0 |
| | | | | oxide | | | | | |
| 15 | Comparative | B | 5.8 | Silicon | 2 | 0.0003 | 151 | 24 | 0.3 |
| | Example | | | oxide | | | | | |
| 16 | Comparative | B | 5.8 | Silicon | 300 | 0.0517 | 151 | 24 | 0.3 |
| | Example | | | oxide | | | | | |
| 17 | Comparative | C | 4.0 | Silicon | 250 | 0.0625 | 190 | 159 | 2.0 |
| | Example | | | oxide | | | | | |
| 18 | Comparative | D | 2.0 | Silicon | 200 | 0.1000 | 185 | 797 | 10.0 |
| | Example | | | oxide | | | | | |

| | Configuration of magnetic bead | | | | |
|---|---|---|---|---|---|
| | Specific surface area | | | | |
| | | Specific | | Specific | |
| | Specific | surface area | | surface area | |
| | Surface | B1 | | B2 | |
| | area A | calculated | | calculated | |
| | measured | based on | | based on | |
| | by gas | volume-based | | number-based | |
| | adsorption | particle size | Ratio | particle size | |
| Sample | method | distribution | A/B1 | distribution | Ratio |
| No. | m²/g | m²/g | — | m²/g | A/B2 |
| 9 | 0.78 | 0.24 | 3.25 | 0.33 | 2.36 |
| 10 | 0.38 | 0.34 | 1.12 | 0.36 | 1.06 |
| 11 | 3.60 | 0.56 | 6.43 | 0.84 | 4.29 |
| 12 | 8.20 | 0.95 | 8.63 | 1.50 | 5.47 |
| 13 | 3.00 | 2.50 | 1.20 | 2.70 | 1.11 |
| 14 | 1.20 | 0.26 | 4.62 | 0.32 | 3.75 |
| 15 | 0.27 | 0.22 | 1.23 | 0.25 | 1.08 |
| 16 | 12.60 | 0.22 | 57.27 | 0.25 | 50.40 |
| 17 | 11.60 | 0.30 | 38.67 | 0.36 | 32.22 |
| 18 | 10.40 | 0.32 | 32.50 | 0.39 | 26.67 |

### 8. Extraction of Nucleic Acid Using Magnetic Bead

In Examples 1 to 12 shown in Table 4, nucleic acids were extracted using the magnetic beads of Sample Nos. 1 to 4. In Comparative Examples 1 to 12 shown in Table 4, nucleic acids were extracted using the magnetic beads of Sample Nos. 5 to 8. Further, in Examples 13 to 18 shown in Table 5, nucleic acids were extracted using the magnetic beads of Sample Nos. 9 to 14. In Comparative Examples 13 to 16 shown in Table 5, nucleic acids were extracted using the magnetic beads of Sample Nos. 15 to 18.

A nucleic acid extraction method is as follows.

First, human genomic DNA was prepared as a nucleic acid model, and lysozyme was prepared as a model of impurities.

Next, the magnetic beads of each of Examples and Comparative Examples were dispersed in pure water and stirred to prepare a magnetic bead suspension. A content of the magnetic bead in the magnetic bead suspension was 53.17 mass %.

Next, reagents other than the nucleic acid were left to stand to reach a room temperature, and then the reagents were charged into a tube in the following order.
Pure water: 65 μL
Nucleic acid dispersion liquid having a concentration of 0.1 μg/μL: 20 μL
Dissolution and adsorption liquid: 750 μL
Lysozyme aqueous solution having a concentration of 10 μg/μL: 15 μL
Magnetic bead suspension: 40 μL

Next, contents of the tube were stirred with a vortex mixer for 10 minutes, then the tube was set at a magnetic stand and allowed to stand for 30 seconds. When the magnetic bead was magnetically trapped, the supernatant was removed.

Next, 900 μL of a washing liquid was added to the tube, followed by stirring with a vortex mixer for 5 seconds and centrifugation. Thereafter, the tube was set at a magnetic stand and allowed to stand for 30 seconds. When the magnetic bead was magnetically trapped, the supernatant was removed. Thereafter, addition of the washing liquid, magnetic trapping, and removal of the supernatant were performed once again.

Next, 900 μL of 70% ethanol aqueous solution was added to the tube, followed by stirring with a vortex mixer for 5 seconds and centrifugation. Thereafter, the contents of the tube were suctioned with a pipette and returned once. Thereafter, the tube was set at a magnetic stand and allowed to stand for 30 seconds. When the magnetic bead was magnetically trapped, the supernatant was removed. Thereafter, addition of the ethanol aqueous solution, magnetic trapping, and removal of the supernatant were performed once again. Next, the tube was subjected to centrifugation, and the remaining supernatant was removed.

Next, 100 μL of pure water was added to the tube, followed by stirring with a vortex mixer to elute the nucleic acid. At this time, a stirring time with the vortex mixer was set to the elution time shown in Tables 4 and 5. Thereafter, the tube was set at a magnetic stand and allowed to stand for 30 seconds. When the magnetic bead was magnetically trapped, an elution liquid containing the nucleic acid was collected into another tube.

### 9. Evaluation of Extracted Nucleic Acid

The nucleic acids extracted in Examples and Comparative Examples were evaluated by the following methods.

### 9.1. Yield of Nucleic Acid

For the elution liquid containing the extracted nucleic acids, the yield of nucleic acids was calculated by the following method.

First, the tube containing the collected elution liquid was set in a spectrophotometer, and a concentration of the nucleic acid in the elution liquid was determined based on an absorbance at a wavelength of 260 nm. Since a nucleotide base has absorption maximum near 260 nm, the absorbance at the wavelength of 260 nm was used to quantify the concentration of the nucleic acid. Then, a collection amount of the nucleic acids was calculated based on the determined concentration, and a ratio of the collection amount to an input amount of the nucleic acid was calculated as the yield. The calculation results are shown in Tables 4 and 5.

The calculated yield of nucleic acid was evaluated in light of the following evaluation criteria. The evaluation results are shown in Tables 4 and 5 as relative evaluations of the yield.
A: The yield of nucleic acid is 80% or more
B: The yield of nucleic acid is 65% or more and less than 80%
C: The yield of nucleic acid is 50% or more and less than 65%
D: The yield of nucleic acid is less than 50%

### 9.2. Purity of Nucleic Acid

An absorbance A260 at a wavelength of 260 nm was measured for an elution liquid containing the extracted nucleic acid by the method shown in 9.1. In addition, an absorbance A280 at a wavelength of 280 nm was also measured.

Next, a ratio A260/A280 of the absorbance A260 to the absorbance A280 was calculated. The calculation results are shown in Tables 4 and 5 as the purity of the nucleic acid.

The calculation results were evaluated in light of the following evaluation criteria. The evaluation results are shown in Tables 4 and 5 as relative evaluations of the purity.
A: The ratio A260/A280 is 1.7 or more and 1.9 or less
B: The ratio A260/A280 is less than 1.7 or more than 1.9

TABLE 4

| | | Nucleic | | | | |
|---|---|---|---|---|---|---|
| | | acid | Evaluation result of magnetic bead | | | |
| | | extraction | | | Purity of | |
| | Magnetic | condition | Yield of | Relative | nucleic | Relative |
| | bead | Elution | nucleic | evaluation | acid (ratio | evaluation |
| Classifi- | Sample | time | acid | of yield | A260/A280) | of purity |
| cation | No. | min(s) | % | — | — | — |
| Example 1 | No. 1 | 10 | 90 | A | 1.8 | A |
| Example 2 | No. 1 | 5 | 85 | A | 1.9 | A |
| Example 3 | No. 1 | 1 | 81 | A | 1.8 | A |
| Example 4 | No. 2 | 10 | 88 | A | 1.9 | A |
| Example 5 | No. 2 | 5 | 87 | A | 1.9 | A |
| Example 6 | No. 2 | 1 | 82 | A | 1.8 | A |
| Example 7 | No. 3 | 10 | 85 | A | 1.8 | A |
| Example 8 | No. 3 | 5 | 75 | B | 1.8 | A |
| Example 9 | No. 3 | 1 | 71 | B | 1.8 | A |
| Example 10 | No. 4 | 10 | 82 | A | 1.8 | A |
| Example 11 | No. 4 | 5 | 73 | B | 1.8 | A |
| Example 12 | No. 4 | 1 | 70 | B | 1.8 | A |
| Comparative | No. 5 | 10 | 62 | C | 1.5 | B |
| Example 1 | | | | | | |
| Comparative | No. 5 | 5 | 55 | C | 1.6 | B |
| Example 2 | | | | | | |
| Comparative | No. 5 | 1 | 43 | D | 1.7 | A |
| Example 3 | | | | | | |
| Comparative | No. 6 | 10 | 72 | B | 1.5 | B |
| Example 4 | | | | | | |
| Comparative | No. 6 | 5 | 55 | C | 1.6 | B |
| Example 5 | | | | | | |
| Comparative | No. 6 | 1 | 42 | D | 1.7 | A |
| Example 6 | | | | | | |
| Comparative | No. 7 | 10 | 32 | D | 1.5 | B |
| Example 7 | | | | | | |
| Comparative | No. 7 | 5 | 25 | D | 1.6 | B |
| Example 8 | | | | | | |
| Comparative | No. 7 | 1 | 19 | D | 1.7 | A |
| Example 9 | | | | | | |
| Comparative | No. 8 | 10 | 68 | B | 1.4 | B |
| Example 10 | | | | | | |
| Comparative | No. 8 | 5 | 52 | C | 1.5 | B |
| Example 11 | | | | | | |
| Comparative | No. 8 | 1 | 41 | D | 1.6 | B |
| Example 12 | | | | | | |

TABLE 5

| | | Nucleic | | | | |
|---|---|---|---|---|---|---|
| | | acid | Evaluation result of magnetic bead | | | |
| | | extraction | | | Purity of | |
| | Magnetic | condition | Yield of | Relative | nucleic | Relative |
| | bead | Elution | nucleic | evaluation | acid (ratio | evaluation |
| Classifi- | Sample | time | acid | of yield | A260/A280) | of purity |
| cation | No. | min(s) | % | — | — | — |
| Example 13 | No. 9 | 5 | 85 | A | 1.9 | A |
| Example 14 | No. 10 | 5 | 75 | B | 1.8 | A |
| Example 15 | No. 11 | 5 | 70 | B | 1.8 | A |
| Example 16 | No. 12 | 5 | 61 | C | 1.7 | A |
| Example 17 | No. 13 | 5 | 71 | B | 1.8 | A |
| Example 18 | No. 14 | 5 | 83 | A | 1.9 | A |
| Comparative | No. 15 | 5 | 25 | D | 1.6 | B |
| Example 13 | | | | | | |
| Comparative | No. 16 | 5 | 53 | C | 1.4 | B |
| Example 14 | | | | | | |
| Comparative | No. 17 | 5 | 54 | C | 1.5 | B |
| Example 15 | | | | | | |
| Comparative | No. 18 | 5 | 55 | C | 1.5 | B |
| Example 16 | | | | | | |

As shown in Tables 4 and 5, it is found that by using the magnetic bead of each Example, a nucleic acid can be extracted at a high yield even when an extraction time is short. It is also found that a high-purity nucleic acid can be extracted by using the magnetic bead of each Example. Based on the above results, it is found that the magnetic bead and the magnetic bead reagent according to the present disclosure can efficiently extract a high-purity biological substance.

What is claimed is:

1. A magnetic bead comprising:
a magnetic metal particle; and
a coating layer covering a surface of the magnetic metal particle and containing an inorganic oxide, wherein
a specific surface area A measured by a gas adsorption method is 0.3 m²/g or more and 10.0 m²/g or less, and
a ratio A/B1 of the specific surface area A to a specific surface area B1 calculated based on a volume-based particle size distribution is 1.00 or more and 9.00 or less.

2. The magnetic bead according to claim 1, wherein
the inorganic oxide is a silicon oxide.

3. The magnetic bead according to claim 1, wherein
a ratio A/B2 of the specific surface area A to a specific surface area B2 calculated based on a number-based particle size distribution is 1.00 or more and 6.00 or less.

4. The magnetic bead according to claim 1, wherein
the magnetic metal particle is made of a Fe-based alloy.

5. The magnetic bead according to claim 4, wherein
the Fe-based alloy has an amorphous structure.

6. The magnetic bead according to claim 1, wherein
a particle diameter D50 at which a cumulative value from a small diameter side in an integrated distribution curve obtained from the volume-based particle size distribution is 50% is 0.5 μm or more and 15 μm or less.

7. The magnetic bead according to claim 6, wherein
a ratio t/D50 of an average thickness t of the coating layer to the particle diameter D50 is 0.0005 or more and 0.2 or less.

8. A magnetic bead reagent comprising:
the magnetic bead according to claim 1; and
a dispersion medium in which the magnetic bead is dispersed.
